(19) United States
(12) Patent Application Publication
(10) Pub. No.: US 2021/0309978 A1
(43) Pub. Date: Oct. 7, 2021

(54) POLYNUCLEOTIDE SEQUENCE ENCODING A LOCAL SOURCED NOVEL ESTERASE

(71) Applicant: ISTANBUL TEKNIK UNIVERSITESI, Istanbul (TR)

(72) Inventors: Havva Esra TUTUNCU, Istanbul (TR); Nevin Gul KARAGULER, Istanbul (TR); Nurgul CELIK BALCI, Istanbul (TR); Melek TUTER, Istanbul (TR)

(73) Assignee: ISTANBUL TEKNIK UNIVERSITESI, Istanbul (TR)

(21) Appl. No.: 17/266,129

(22) PCT Filed: Aug. 2, 2019
(86) PCT No.: PCT/TR2019/050646

§ 371 Date: Feb. 5, 2021

(30) Foreign Application Priority Data
Aug. 9, 2018 (TR) 2018/11617

(51) Int. Cl. C12N 9/16

(52) U.S. Cl. CPC C12N 9/16

(57) ABSTRACT

An esterase enzyme is provided. A polynucleotide sequence is shown in SEQ ID NO. 1. A sequence number of a protein encoded by the polynucleotide sequence is shown in SEQ ID NO. 2. The polynucleotide sequence is obtained as a result of a polymerase chain reaction with a first primers having a nucleotide sequence shown in SEQ ID NO. 3 and a second primer having a nucleotide sequence shown in SEQ ID NO. 4.

### CROSS REFERENCE TO THE RELATED APPLICATIONS

This application is the national stage entry of International Application No. PCT/TR2019/050646, filed on Aug. 2, 2019, which is based upon and claims priority to Turkish Patent Application No. 2018/11617, filed on Aug. 9, 2018, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBAB059_Sequence_Listing.txt, created on Feb. 28, 2021 and is 4,443 bytes in size.

### TECHNICAL FIELD

The invention is related to finding a novel polynucleotide sequence (gene), revealing a novel polypeptide (enzyme/protein) encoded by this polynucleotide sequence and the production and purification of this polypeptide and defining the function thereof.

The invention is particularly related to establishing a novel esterase enzyme that can be used in the industry and that is obtained by molecular biological methods from the microflora of Ac1göl (Aci Lake) located in Denizli.

The invention is related to the production and usage of host cells and esterases that comprise vectors and esterase enzymes and artificially obtained nucleic acid.

### BACKGROUND

Microorganisms have been used for a long time in the production of biomolecules that are significantly important to the industry. Approaches depending on traditional culture as a fundamental strategy have been used for many years in order to convey the potentials of these microorganisms. However, only a very small amount of microorganisms in the world can be cultured. This means that we cannot benefit from the remaining large amounts of microorganisms by using traditional culturing methods. Therefore, the metagenomic approach comes in handy at this point. Initially in 1998, “metagenome” has been defined as the “total genome of soil microflora” and its definition has then been extended to cover all environmental medium in which microbic activities such as human and animal excretion take place, such as sea, lakes, thermal springs, acid mining drainage sites and wastewater sites etc. (Handelsman et al. 1998; Venter et al. 2004; Hardeman and Sjöling, 2007; Rhee et al. 2005; Tirawongsaroj et al. 2008; Tyson et al. 2004; Jones et al. 2011; Roh and Vilatte, 2008; Althani et al. 2016; Ilmberger et al. 2014; Amimo et al. 2016). In studies where the metagenomic approach has been used, the cells located inside the environmental samples are used directly without the need for separating them from the medium they are located in and without the need for identifying these cells. By this means a maximum variety of genomic material can be obtained.

Esterase and lipase which are lipolytic enzymes are present in the hydrolase group enzymes that form the majority of the enzymes that are used in the industry. In the energy industry, they are used in the esterification of free fatty acids with alcohol in the production of biodiesel or the transesterification of triacylglycerides. Exemplary applications in the food industry comprise the enhancement of aroma and taste, enhancement of dough characteristics, and improving EPA and DHA rates in fish oil. Esterase and lipases are also provided as enzymes that play a critical role in catalyzing several different reactions in the detergent, pharmaceutical and cosmetic fields. Detailed reviews related to the current status of lipolytic enzymes can be found in the literature (Houde et al, 2004; Panda and Gowrishankar, 2005; Ribeiro et al, 2011; Borrelli and Trono, 2015).

Several lipase and esterase enzymes have been isolated from different organisms until today and they have been characterized and some of these have been industrially produced and used. More active and robust enzymes have been obtained using various protein engineering techniques and extremophilic organisms. However, enzymes that have new characteristics are still required (Hasan et al, 2009; Borrelli and Trono, 2015). When we contemplate industrially, when high amounts of the enzyme are required, we realize that production costs are a significant limiting factor. Moreover, enzyme inhibition due to glycerol, small chain alcohols or other agents appears to be a problem that needs to be overcome (Ribeiro et al, 2011). Daiha et al. (2015) have prepared a report which compiles the fields of application, patents and research articles that comprise the usage and production of these enzymes since the 1930s, which is the date that these lipolytic enzymes have been initially discovered. They have studied the necessity of novel lipolytic enzymes by using a method called “Technological life cycle”. According to analysis results, it has been determined that novel lipolytic enzymes are still required in the industry.

In the Chinese patent document numbered CN102286441 of the prior art, low-temperature esterase enzyme, encoding gene and usage thereof is disclosed. The novel esterase gene subject to the invention has been extracted from the metagenomic library of the abyssal deposit taken from the Pacific Ocean and it has been determined that the protein encoded by the gene has enzymatic features.

In the Chinese patent document numbered CN101402947 of the prior art, a novel esterase gene named Est_p1 and the recombination expression system of this gene is disclosed. The gene has been encoded from the metagenomic library of the sludge 100 meters below the South China Sea. The gene has a length of 891 bp and which encodes a protein at an amino acid length of 296, has been produced using a recombination expression system called pET28a-Est_p1/BL21. The molecular weight of the target protein is 33.5 kD. In the activity assay carried out with pNP-butyric ester, it has been proved that Est_p1 is an alkali esterase. Est_p1 has strong catalytic activity for short chain esters and can be used in esterification and transesterification industries.

In the Korean patent document numbered KR101400903 of the prior art a novel esterase that has been isolated from the compost metagenomic library has been disclosed. In the invention of an encoding gene, a recombinant vector comprising the gene, a transformant transformed with an expression vector and a production method for esterase is described. Esterase displays perfect activity between pH 5 and 10 and preferably at pH 8. It maintains esterase activity between 0 to 60° C.

Although there are several lipolytic enzymes present in the art, novel enzymes are still required. Therefore, the development of a polynucleotide sequence encoding a local sourced and novel esterase is planned. As it has been mentioned above, methods depending on culturing limit the variety of microorganisms, and therefore, beneficial biomolecule numbers obtained from them. In the study that has been developed a metagenomic method has been used and this problem has been overcome.

The metagenomic method has been used for several years in order to reach this aim. The most important characteristic of this invention is that the polynucleotide sequence obtained with the metagenomic method has been revealed for the first time with this invention.

### SUMMARY

The invention aims to find a novel polynucleotide sequence (gene), create a novel polypeptide (enzyme/protein) encoded by this polynucleotide sequence and to produce and purify this polypeptide and define the function thereof.

Another aim of the invention is to establish a novel esterase enzyme that can be used in the industry and that is obtained by molecular biological methods from the microflora of Ac1göl (Aci Lake) located in Denizli.

Another aim of the invention is to provide an esterase enzyme that exhibits high activity at low temperatures, metal tolerance, and has usage potential in the industry due to its organic solution stability.

### DETAILED DESCRIPTION OF THE EMBODIMENTS

The enzyme subject to the invention has been obtained by means of the process steps mentioned below.

1. Metagenomic DNA isolation: In this invention, first of all, samples have been collected from Ac1göl under sterile conditions, pre-enrichment has been applied in order to increase the cell number and metagenomic DNA has been obtained isolated.

2. Primer design and degenerate polymerase chain reaction: The polynucleotide sequences which encode lipolytic proteins from halophilic microorganisms found in the natural flora of Ac1göl have been obtained from the NCBI database and they have been sequenced as a list used bioinformatic tools and the conserved sites have been determined. Degenerate primers have been designed to target these sites and a part of the polynucleotide sequence has been obtained by a polymerase chain reaction.
CODEHOP strategy has been used in primer design (Rose et al., 1998). According to this strategy, the 5′ ends of the primers were perfectly compatible with the target sequence while the 3′ end comprises degenerated nucleotides that enable binding of different nucleotides during DNA replication. Several studies are present where primers having such structures are used. However, the primers that have been designed within the scope of this study, do not belong to any kind of study that has been carried out before and it has been designed to be specific to the targeted sequence in this study. The primer sequences used are as follows:

HCABAF:
5′-GGGCCATAGCATGGGNGGNAARG-3′
(as shown in SEQ ID NO: 3)
HCABUR:
5′-GGTTGATCGGCATGCARCCARTG-3′
(as shown in SEQ ID NO: 4)

3. Genome walking: All of the partial polynucleotide sequences has been clarified. The obtained polynucleotide sequence (gene) has been shown in SEQ ID NO: 1, and the protein encoded by this gene has been shown in SEQ ID NO: 2.

4. Bioinformatic analysis of the sequence: The novel gene sequence that has been obtained has been compared in terms of similarity with the sequences that have been discovered before, from the present sequences in the NCBI database.

5. Cloning of the gene, protein expression and purification: After all the gene sequence has been obtained, the gene has been reproduced by means of polymerase chain reaction using primers that contain the restriction sites of EcoRI and HindIII. Both the reproduced gene and the pET-28a(+) vector has been cut off with the same enzymes and the vector and the insert has been bound together and it has been transformed into E. coli C43(DE3) cells which is an expression cell.
After the polymerase chain reaction product was purified, it has been digested with 5 U EcoRI and 5 U HindIII restriction enzymes for 2.5 hours at 37° C. The enzymes have been inactivated for 20 minutes at 80° C. In order to prepare the vector, 1 μg of vector has been cut for 2 hours at 37° C. with 1 U of EcoRI and 1 U of HindIII. In order to remove the phosphate groups at the binding regions, it has been incubated for 1 hour at 1 37° C. with 1.5 Uphosphatase and the enzyme has been inactivated for 10 minutes at 80° C. Following the digestion reaction, the gene and the vector to be cloned have been purified from agarose gel and they have been made ready for ligation.
The aim of transforming the ligated vector into E. coli C43(DE3) cells is to ensure the production of the enzyme. The gene and the vector that is to be cloned are incubated for 18 hours at 15° C. with T4 DNA ligase enzyme and ligated. 5 μl of ligation product has been transformed into 50 μl of competent host cells (E. coli C43(DE3)) and following 30 minutes of incubation on ice, it has been subjected to heat shock for 45 hours at 42° C. By this means, the vector is inserted into the cell.
Starter culture having a volume of 50 mL has been inoculated into the 1 liter LB liquid medium containing 40 μg/μl kanamycin for protein expression. Growth has been monitored until the optical density value (OD600 nm) reached to 0.8, and following this, induction with 0.1 mM isopropyl-β-D-thiogalactopyranoside (IPTG) has been carried out at 30° C. for 6 hours. After incubation, the cells have been collected with centrifuge and have been stored at −20° C. until they were used.
Protein purification process has been carried out using Ni-NTA agarose according to the His-Tag protein purification method. The protein that has been purified has been viewed with Coomassie Brilliant Blue G-250 after SDS-PAGE analysis.

6. Standard activity test: Activity has been measured based on the pNP measurement that is obtained by the hydrolysis of the paranitfrophenol (pNP) esters by the enzyme (Winkler and Stuckmann, 1979). The final concentration of the enzyme and substrate have been 1 μM and 1 mM respectively and absorbance measurements have been taken at 410 nm. The activity has been calculated by means of the below formula and one unit of activity has been defined as the enzyme amount which produces 1 μmol product in 1 minute.

Activity (U/L)=[(ΔAbs/t)/(ε*d)]*106*Vt/Vs
ΔAbs=Absorbance change; t=time (minute); ε=extinction coefficient (M−1 cm−1); d=light path (cm); Vt=total reaction volume; Vs=enzyme volume used in reaction.

7. Substrate specificity test: pNP esters (pNP-butyrate, pNP-hexanoate, pNP-octanoate, pNP-decanoate, pNP-dodecanoate and pNP-hexadecanoate) having different carbon chain lengths have been used in order to measure the reaction of the enzyme against different substrates and Michaelis Menten kinetic parameters have been calculated. The reactions have been set up to comprise 0.031, 0.0625, 0.125, 0.25, 0.5, 0.75, 1, 1.25, 1.5, 2, 2.5 and 3 mM substrates and GraFit 7.0 program was used for calculations.

8. Measuring the activity and stability of the enzyme under different conditions: Reactions have been carried out in buffer solutions (50 mM citrate/phosphate buffer, pH 6; 50 mM Tris-HCl buffer, pH 7, 8 and 8.5; 50 mM glycine-NaOH buffer, pH 9, 9.5 and 10) having different pH values in order to determine the optimum pH value in which the enzyme works. Moreover, in order to measure the pH stability of the enzyme, the enzyme has been incubated for 24 hours before reaction in these buffer solutions and the activity value remaining at the end of the incubation has been calculated.

The reaction has been carried out under different temperatures (5° C.-70° C.) in order to determine the optimum temperature in which the enzyme works efficiently, and the activity has been measured. In order to determine the thermal stability of the enzyme, the enzyme has been incubated for 20 minutes between 25° C.-80° C. temperature and the remaining activity has been measured according to standard tests.

TABLE 1
The effect of temperature on the enzyme

| Effect of temperature | | Effect of temperature | |
|---|---|---|---|
| on activity | | on stability | |
| Temperature | Activity | Temperature | Activity |
| (° C.) | (%) | (° C.) | (%) |
| 5 | 46.6 ± 6.1 | 25 | 100 ± 1.4 |
| 10 | 53.8 ± 7.4 | 30 | 104.3 ± 4.3 |
| 15 | 63.1 ± 5.1 | 35 | 99.9 ± 0.6 |
| 20 | 76.9 ± 4.4 | 40 | 98.6 ± 0.8 |
| 25 | 93.1 ± 6.3 | 45 | 95.1 ± 2 |
| 30 | 100 ± 4.8 | 50 | 92.8 ± 2.5 |
| 35 | 94.4 ± 1.5 | 55 | 95.9 ± 5.4 |
| 40 | 82.4 ± 2.8 | 60 | 27.3 ± 4.8 |
| 45 | 22.5 ± 0.8 | 65 | 2.8 ± 0.4 |
| 50 | 17.7 ± 0.3 | 70 | 4.5 ± 0.6 |
| 55 | 14.4 ± 4.5 | 75 | 2.3 |
| 60 | 12.9 ± 2.9 | 80 | 2.2 ± 0.1 |
| 65 | 11.1 ± 4.2 | | |
| 70 | 8.2 ± 0.8 | | |

The effect of sodium chloride on the activity (5%, 10%, 15%, 20% and 25% NaCl) and the stability (10%, 15%, 20% and 25% NaCl) of the enzyme has been determined similarly. The enzyme has been incubated in a buffer solution comprising sodium chloride at the defined amounts for 24 hours during stability tests.

TABLE 2
The effect of sodium chloride on the enzyme

| The effect of sodium | | The effect of sodium | |
|---|---|---|---|
| chloride on activity | | chloride on stability | |
| Concentration | Activity | Concentration | Activity |
| (%) | (%) | (%) | (%) |
| 1 | 100 ± 1.9 | 10.0 | 100 ± 4.8 |
| 5 | 90.2 ± 2.1 | 15.0 | 99 ± 1.8 |
| 10 | 79.2 ± 5 | 20.0 | 102.7 ± 1 |
| 15 | 52 ± 5.3 | 25.0 | 101.4 ± 4.5 |
| 20 | 39.2 ± 6.3 | | |

Additionally, the effect of different solutions (hexane, chloroform, acetone, dimethyl formamide (DMF), dimethyl sulphoxide (DMSO), 2-propanol, butanol, 1-propanol, ethanol, methanol and glycerol) on the activity and stability of the enzyme have been measured. In the activity tests, 10% solution has been added into the reaction and in the stability tests the enzyme has been incubated for 24 hours in a buffer solution containing 15% and 30% solution.

TABLE 3
The effect of solutions on the enzyme

| Effect of solutions | | Effect of solutions on stability | | |
|---|---|---|---|---|
| on activity | | | Activity (%) | Activity (%) |
| Solution | Activity (%) | Solution | 15% Solution | 30% Solution |
| Hexane | 22.6 ± 4.9 | Hexane | 113.6 ± 8.5 | 100.2 ± 4.9 |
| Chloroform | 5.1 ± 0.8 | Chloroform | 97.9 ± 1.4 | 100.2 ± 5.6 |
| Acetone | 36 ± 4.9 | Acetone | 113.7 ± 2.5 | 85.2 ± 5.6 |
| DMF | 33.2 ± 7.0 | DMF | 121.3 ± 1.6 | 105.3 ± 1.6 |
| DMSO | 92 ± 4.3 | DMSO | 106.1 ± 0.9 | 102.2 ± 4.8 |
| 2-Propanol | 21.4 ± 1.6 | 2-Propanol | 104.3 ± 1.2 | 62.3 ± 4.3 |
| Butanol | 3.3 ± 3.3 | Butanol | 97.1 ± 3.3 | 104.9 ± 2.4 |
| 1-Propanol | 2.6 ± 2.4 | 1-Propanol | 85.7 ± 2.6 | 23.3 ± 5.4 |
| Ethanol | 45.1 ± 0.3 | Ethanol | 105.9 ± 4.7 | 98.2 ± 0.5 |
| Methanol | 81.1 ± 1.1 | Methanol | 108.5 ± 0.2 | 99.4 ± 6.6 |
| Glycerol | 104.2 ± 6.2 | Glycerol | 113.3 ± 1.8 | 106.9 ± 3.1 |
| Control | 100 ± 0.1 | Control | 100 ± 4.6 | 100 ± 4.6 |

The effect of metal ions (MgCl₂, FeSO₄, KCl, CaCl₂), ZnSO₄, Al₂SO₄, CuSO₄, CoCl₂, NiCl₂, MnSO₄, AgNO₃), some detergents (sodium dodecyl sulphate (SDS), cetrimonium bromide (CTAB), Triton X-100, Tween-80, Nonidet-P40) and inhibition agents (phenylmethanesulfonyl fluoride (PMSF), dithiothreitol (DTT), ethylene diamine tetraacetic acid (EDTA) and ethylene glycol tetraacetic acid (EGTA) on the enzyme activity has been examined.

TABLE 4
Effect of metals on activity

| | Activity (%) | Activity (%) |
|---|---|---|
| Metal | (1 mM metal) | (5 mM metal) |
| MgCl₂ | 130.2 ± 0.6 | 104.6 ± 3 |
| FeSO₄ | 115.7 ± 2.3 | 0.0 |
| KCl | 127.9 ± 3.9 | 98.2 ± 3.4 |
| CaCl₂ | 130.2 ± 1.7 | 105.1 ± 1.4 |
| ZnSO₄ | 6.1 ± 3.1 | 1.3 ± 1.2 |
| Al₂SO₄ | 91.2 ± 9.8 | 0.0 |
| CuSO₄ | 117.5 ± 5 | 1.4 ± 1.1 |
| CoCl₂ | 112.7 ± 3.4 | 3.6 ± 1.0 |
| NiCl₂ | 110.4 ± 6.1 | 0.5 |
| MnSO₄ | 130.4 ± 0.4 | 110.3 ± 3.5 |
| AgNO₃ | 31.6 ± 6.5 | 0.0 |
| Control | 100 ± 3.6 | |

TABLE 5
Effects of inhibitors on activity

| | Activity (%) | Activity (%) |
|---|---|---|
| Inhibitor | (1 mM inh.) | (5 mM inh.) |
| PMSF | 1.3 ± 0.1 | 0.2 |
| DTT | 36.7 ± 2.4 | 25.1 ± 0.8 |
| EDTA | 89.6 ± 1.7 | 66.5 ± 1.2 |
| EGTA | 100.2 ± 2.1 | 87.6 ± 3.9 |
| SDS | 85.6 ± 2.9 | 5.0 ± 0.8 |
| CTAB | 91 ± 6.6 | 1.3 ± 0.3 |
| Triton X-100 | 105.3 ± 2.4 | 87.7 ± 3.2 |
| Tween-80 | 107.2 ± 7.9 | 80.7 ± 2.2 |
| NP-40 | 101.9 ± 6.8 | 89.1 ± 1.5 |
| Control | 100 ± 3.5 | |

The obtained novel polynucleotide sequence encodes a functional polypeptide sequence. It has been noted that polypeptide had esterase activity following its successful production, purification and characterization and that it has some characteristics that are important in terms of industry. These characteristics have been listed below.

1. The polynucleotide sequence that has been obtained is formed of 260 amino acids that have a length of 783 base pairs (SEQ ID NO: 1). The gene sequence resembles a part of the Halolamina sediminis genome by 75% (NCBI Accession Number: CP018139.1). The amino acid sequence resembles the esterase of Halomonas gudaonensis by 91% (SDJ79551.1). As a result, it has been noted that a novel enzyme that has not been identified before from an environmental sample, has been cloned successfully.

TABLE 6
the sequences that resemble the est_ag
polynucleotide sequence the most.

| Accession | | Total | | |
|---|---|---|---|---|
| number | Description | score | CQ | ID |
| CP018139.1 | Halolamina sediminis strain Hb3, | 307 | 98% | 75% |
| | complete genome | | | |
| FN869568.2 | Halomonas elongato DSM 2581, | 271 | 100% | 73% |
| | complete genome | | | |
| CP014226.1 | Halomonas chromatireductens strain | 174 | 98% | 68% |
| | AGD 8-3, complete genome | | | |
| 1.N813019.1 | Halomonas sp. R57-5 genome | 163 | 100% | 68% |
| | assembly HalomonasR57-5, | | | |
| | chromosome: 1 | | | |
| CP019326.1 | Halomonas sp. 1513, | 152 | 88% | 69% |
| | complete genome | | | |
| 1.T670847.1 | Halomonas subglaciescola strain | 141 | 98% | 68% |
| | ACAM 12 genome assembly, | | | |
| | chromosome: 1 | | | |
| CP011052.1 | Halomonas sp. KO116, | 113 | 99% | 66% |
| | complete genome | | | |
| 1.T593974.1 | Halomonas sp. HL-93 | 84.2 | 97% | 65% |
| | genome assembly, chromosome: 1 | | | |
| CP017114.1 | Cohetia marina strain JCM 21022, | 82.4 | 45% | 71% |
| | complete genome | | | |

2. The polynucleotide sequence (gene) which encodes the enzyme has been transferred to the pET-28a(+) vector which enables the production of protein in high yields using suitable molecular biological techniques. This vector that has been formed is transferred to E. coli (C43) cells which are expression cells and high amounts of protein have been expressed. Recombinant produced protein has been obtained in a pure manner using the His-tag purification method. By this means the re-production of protein has been enabled. It has been determined that the obtained protein amount was generally around 30 mg per 1 liter of the culture used.

3. It has been determined that the enzyme worked best at pH 9 value. The enzyme has maintained its stability up to pH 10. Being functional in alkali medium is an important feature in the usage of such enzymes in detergent formulations (Lopez et al, 2014).

TABLE 7
Effect of pH on the enzyme

| The effect of pH on activity | | The effect of pH on stability | |
|---|---|---|---|
| pH | Activity (%) | pH | Activity (%) |
| 6 | 10.3 ± 4.1 | 4 | 2.7 |
| 7 | 68.8 ± 8.2 | 5 | 3.9 ± 1.1 |
| 8 | 77.1 ± 6.8 | 6 | 9.9 ± 0.8 |
| 8.5 | 84 ± 5.2 | 7 | 85.9 |
| 9 | 100 ± 1.3 | 8 | 100.3 ± 0.6 |
| 9.5 | 84.9 ± 1.5 | 8.5 | 98.9 ± 5.3 |
| 10 | 20 ± 8.1 | 9 | 100 ± 2.4 |
| | | 9.5 | 104.4 ± 3.5 |
| | | 10 | 108.6 ± 0.3 |

4. The enzyme has shown the best activity at 30° C. and has maintained 47% of its maximum activity in 5° C. and has maintained 63% of its activity at 15° C. This feature is very important in order to reduce energy costs in the biotechnology industry. While high temperature is required in order to obtain the desired products in some reactions that do not contain enzymes, the same product can be obtained under low temperatures with enzymes that show function in cold temperatures. Such enzymes are required in the production of detergents and food, for the bioremediation of seas or lands, or reactions where volatile compounds that are susceptible to temperatures are used (Lopez et al, 2014).

5. When the thermal stability was examined it has been observed that the enzyme maintained the stability up to 55° C.

6. When the activity of the enzyme has been examined in the presence of 10% solvents, it has been noted that it showed high activity in the presence of DMSO or methanol (92% and 81% respectively). The activity has increased by 4% in the presence of glycerol. It has also been determined that the enzyme has a stable structure in stability tests carried out in the presence of a solvent solution. The reactions that are carried out in the presence of an organic solution have advantages such as high solubility of hydrophobic materials in such mediums, not having any by-products due to the exclusion of water molecules and thermodynamic balance which shifts from hydrolysis to synthesis (Salihu and Alam, 2015).

7. The activity of the enzyme has been measured in the presence of various metal ions and it has been noted that it showed much higher activity according to several similar enzymes in literature.

TABLE 8
Comparison of the enzyme with other enzymes in
literature in terms of metal tolerance

| | Glogauer | Mohamed | Castilla | Zhang et | |
|---|---|---|---|---|---|
| Metal | et al, | et al, | et al, | al, | This |
| ion | 2011^{a} | 2013 | 2017 | 2017 | study |
| mg²⁺ | 65 | 91 | 160 | 97 | 130 |
| K⁺ | 122 | N/A | 90 | 108 | 128 |
| Ca²⁺ | 73 | 95 | 110 | 95 | 130 |
| Co²⁺ | 54 | 104 | 50 | 85 | 113 |
| Cu²⁺ | N/A | 95 | 60 | 82 | 118 |
| Fe²⁺ | 33 | N/A | 55 | 85 | 116 |
| Mn²⁺ | 47 | 112 | 75 | 76 | 130 |
| Ni²⁺ | 66 | N/A | 75 | 68 | 110 |
| Zn²⁺ | 45 | 85 | 160 | 25 | 6 |
| Al³⁺ | 45 | N/A | N/A | N/A | 91 |
| Ag⁺ | 8 | N/A | N/A | N/A | 32 |

By means of the invention:
An enzyme, whose sequence and characteristics have been determined for the first time and a novel gene encoding this enzyme has been discovered from the local microflora in Turkey,
an esterase enzyme that has not been identified before having potential usage in the industry due its features such as exhibiting high activity at low temperatures, metal tolerance, organic solution stability has been provided,
this enzyme was not only identified it was also transferred into suitable vector and host cells in order for the enzyme to be able to be produced with a high yield so that it is suitable for industrial production.

### REFERENCES

Ithani, A. A., Marei, H. E., Hamdi, W. S., Nasrallah, G. K., El Zowalaty, M. E., Al Khodor, S., Cenciarelli, C. (2016). Human Microbiome and its Association With Health and Diseases. Journal of Cellular Physiology, 231(8), 1688-1694.
Amimo, J. O., El Zowalaty, M. E., Githae, D., Wamalwa, M., Djikeng, A., & Nasrallah, G. K. (2016). Metagenomic analysis demonstrates the diversity of the fecal virome in asymptomatic pigs in East Africa. Archives of Virology, 161(4), 887-897.
Borrelli, G. M., & Trono, D. (2015). Recombinant lipases and phospholipases and their use as biocatalysts for industrial applications. International Journal of Molecular Sciences, 16(9), 20774-20840.
Castilla, A., Panizza, P., Rodriguez, D., Bonino, L., Diaz, P., Irazoqui, G., & Rodriguez Giordano, S. (2017). A novel thermophilic and halophilic esterase from Janibacter sp. R02, the first member of a new lipase family (Family XVII). Enzyme and Microbial Technology, 98, 86-95.
Daiha, G. K., Angeli, R., De Oliveira, S. D., & Almeida, R. V. (2015). Are lipases still important biocatalysts? A study of scientific publications and patents for technological forecasting. PLoS ONE, 10(6), 1-20.
Glogauer, A., Martini, V. P., Faoro, H., Couto, G. H., Müller-Santos, M., Monteiro, R. A., Krieger, N. (2011). Identification and characterization of a new true lipase isolated through metagenomic approach. Microbial Cell Factories, 10(1), 54.
Handelsman, J., Rondon, M. R., Brady, S. F., Clardy, J., & Goodman, R. M. (1998). Molecular biological access to the chemistry of unknown soil microbes: a new frontier for natural products. Chemistry & Biology, 5(10), R245-R249.
Hårdeman, F., & Sjöling, S. (2007). Metagenomic approach for the isolation of a novel low-temperature-active lipase from uncultured bacteria of marine sediment. FEMS Microbiology Ecology, 59(2), 524-534.
Houde, A., Kademi, A., & Leblanc, D. (2004). Lipases and Their Industrial Applications: An Overview. Applied Biochemistry and Biotechnology, 118(1-3), 155-170.
Ilmberger, N., Güllert, S., Dannenberg, J., Rabausch, U., Torres, J., Wemheuer, B., Streit, W. R. (2014). A comparative metagenome survey of the fecal microbiota of a breast- and a plant-fed Asian elephant reveals an unexpectedly high diversity of glycoside hydrolase family enzymes. PLoS ONE, 9(9), 1-12.
Jones, D. S., Albrecht, H. L., Dawson, K. S., Schaperdoth, I., Freeman, K. H., Pi, Y., Macalady, J. L. (2011). Community genomic analysis of an extremely acidophilic sulfur-oxidizing biofilm. The ISME Journal, 6(1), 158-170.
Lopez, O., Cerdán, M. E., & González Siso, M. I. (2014). New extremophilic lipases and esterases from metagenomics. Current Protein & Peptide Science, 15(5), 445-55.
Mohamed, Y. M., Ghazy, M. A., Sayed, A., Ouf, A., El-Dorry, H., & Siam, R. (2013). Isolation and characterization of a heavy metal-resistant, thermophilic esterase from a Red Sea Brine Pool. Scientific Reports, 3, 1-8.
Panda, T., & Gowrishankar, B. S. (2005). Production and applications of esterases. Applied Microbiology and Biotechnology, 67(2), 160-169.
Rhee, J. K., Ahn, D. G., Kim, Y. G., & Oh, J. W. (2005). New thermophilic and thermostable esterase with sequence similarity to the hormone-sensitive lipase family, cloned from a metagenomic library. Applied and Environmental Microbiology, 71(2), 817-825.
Ribeiro, B. D., Castro, A. M. de, Coelho, M. A. Z., & Freire, D. M. G. (2011). Production and Use of Lipases in Bioenergy: A Review from the Feedstocks to Biodiesel Production. Enzyme Research, 2011, 1-16.
Roh, C., & Villatte, F. (2008). Isolation of a low-temperature adapted lipolytic enzyme from uncultivated micro-organism. Journal of Applied Microbiology, 105(1), 116-123.
Rose, T. M., Schultz, E. R., Henikoff, J. G., Pietrokovski, S., McCallum, C. M., & Henikoff, S. (1998). Consensus-degenerate hybrid oligonucleotide primers for amplification of distantly related sequences. Nucleic Acids Research, 26(7), 1628-1635.
Salihu, A., Alam, M. Z (2015) Solvent tolerant lipases: A review, Process Biochem. 50 86-96. doi:10.1016/j.procbio.2014.10.019.
Tirawongsaroj, P., Sriprang, R., Harnpicharnchai, P., Thongaram, T., Champreda, V., Tanapongpipat, S., . . . Eurwilaichitr, L. (2008). Novel thermophilic and thermostable lipolytic enzymes from a Thailand hot spring metagenomic library. Journal of Biotechnology, 133(1), 42-49.
Tyson, G. W., Chapman, J., Hugenholtz, P., Allen, E. E., Ram, R. J., Richardson, P. M., . . . Banfield, J. F. (2004). Community structure and metabolism through reconstruction of microbial genomes from the environment. Nature, 428(6978), 37-43.
Venter, J. C., K. Remington, J. F. Heidelberg, A. L. Halpern, D. Rusch, J. A. Eisen, D. Wu, I. Paulsen, K. E. Nelson, W. Nelson, D. E. Fouts, S. Levy, A. H. Knap, M. W. Lomas, K. Nealson, O. White, J. Peterson, J. Hoffman, R. Parsons, H. Baden-Tillson, C. Pfannkoch, Y. H. Rogers, and H. O. Smith. 2004. Environmental genome shotgun sequencing of the Sargasso Sea. Science 304:66-74.
Winkler, U. K., & Stuckmann, M. (1979). Glycogen, hyaluronate, and some other polysaccharides greatly enhance the formation of exolipase by Serratia marcescens. Journal of Bacteriology, 138(3), 663-670.
Zhang, Y., Hao, J., Zhang, Y. Q., Chen, X. L., Xie, B.-B., Shi, M., . . . Li, P.-Y. (2017). Identification and Characterization of a Novel Salt-Tolerant Esterase from the Deep-Sea Sediment of the South China Sea. Frontiers in Microbiology, 8(March), 1-10.

What is claimed is:

1. A polynucleotide sequence encoding an esterase enzyme, wherein the polynucleotide sequence is shown in SEQ ID NO. 1.

2. The polynucleotide sequence according to claim 1, wherein a sequence of a protein encoded by the polynucleotide sequence is shown in SEQ ID NO. 2.

3. The polynucleotide sequence according to claim 1, wherein the polynucleotide sequence is obtained as a result of a polymerase chain reaction with a first primer having a nucleotide sequence shown in SEQ ID NO. 3 and a second primer having a nucleotide sequence shown in SEQ ID NO. 4.

4. An esterase enzyme production method, comprising the steps of:
collecting halophilic microorganisms,
obtaining a culture by increasing cell amounts in a medium of the halophilic microorganisms,
obtaining a metagenomic DNA from the culture;
finding polynucleotide sequences for encoding lipolytic proteins of the halophilic microorganisms from an NCBI database, determining protected regions and designing degenerated primers shown in SEQ ID NO. 3 and SEQ ID NO. 4 to target the protected regions,
obtaining a part of a polynucleotide sequence shown in SEQ ID NO. 1 as a result of a polymerase chain reaction with the degenerated primers,
obtaining the polynucleotide sequence shown in SEQ ID NO. 1 by clarifying an entire partial polynucleotide sequence,
reproducing the polynucleotide sequence shown in SEQ ID NO. 1, by the polymerase chain reaction using the degenerated primers shown in SEQ ID NO. 3 and SEQ ID NO. 4, wherein the degenerated primers contain restriction sites of gene EcoRI and HindIII,
cutting of a product obtained as a result of the polymerase chain reaction after the product is purified, with 5 U EcoRI and 5 U HindIII restriction enzymes for 2.5 hours at 37° C. and inactivating the 5 U EcoRI and 5 U HindIII restriction enzymes for 20 minutes at 80° C.,
obtaining a vector by cutting for 2 hours at 37° C. with 1 U EcoRI and 1 U HindIII,
incubating the vector in order to remove phosphate groups at binding regions of the vector for 1 hour at 37° C. with 1.5 U phosphatase enzyme,
inactivating the 1.5 U phosphatase enzyme for 10 minutes at 80° C. and purifying the polynucleotide sequence and the vector following cutting reactions from an agarose gel and making the polynucleotide sequence and the vector ready for binding,
transforming the vector and an insert after binding the vector and the insert into E. coli C43(DE3) cells,
incubating the polynucleotide sequence and the vector to be cloned for 18 hours at 15° C. with T4 DNA ligase enzyme in order to bind the polynucleotide sequence and the vector,
adding 5 μl of a ligation product to a competent 50 μl cell for a transformation into host cells, wherein the host cells are E. coli C43(DE3) and, after 30 minutes of incubation on ice, inserting the vector prepared by subjecting the vector to a heat shock for 45 hours at 42° C., into the competent 50 μl cell,
inoculating a starter culture having a volume of 50 mL into a 1 liter LB liquid medium comprising 40 μg/μl kanamycin for a protein expression,
tracking of a growth of the starter culture until an optical density value of the starter culture reaches 0.8,
and carrying out an induction process with 0.1 mM isopropyl-β-D-thiogalactopyranoside (IPTG) at 30° C. for 6 hours.


SEQUENCE LISTING (4 sequences)

SEQ ID NO: 1 - DNA, 783 bp, Unknown
  Metagenomic Sequence
atgaccgtcgatctcaaccatttggacacgggaggggagggcatccccctcgtcgtgatacacgggctgctgggcagcgccgacaactggcgctcccacgtcaagcagtggcagcaacagtgccgtgtcgtcgccgtcgatctgcgcaaccacggtcgctcgccccatgccgacggcatgagctatgctgccatggccgctgatttggaggcgctcctcgatcggctgggtattgagcgtgcccatttgctggggcactccatgggcggcaaagtcgcgatcagcgtggcgcggctccacccgcagcgggttgcctcattgatcgtcgccgatatcgctccacaagcgtacggacacgaccatgacgcggtattcgccggcctgcgacgggtcgagcaggggcagcccggcaatcgccgcgaagccgatgcgctgttggcagagcacgtggaagaacgcgccgttcggctgtttctcgccaccaacctggagcgcgatgatgatggcatattgcggctgcgaatcgggctcgatgaaatcgagggtgactacgacgctatcatgcaggcgccggcaggagagggcagctatgacggcccggtcttggtgctacgcggcgaccgctctccctatgtgaccgacgccatgctgccaaggcttcaccaggtgttaccgcaagcgcaactggtgaccttggaaaacgccggtcactggctgcatgccgagcagcccgaggcattccaggctgccgtcaatgatttcctcgcctccctcgaggcgtga

SEQ ID NO: 2 - protein, 260 aa, Unknown
  Metagenomic Sequence
MTVDLNHLDTGGEGIPLVVIHGLLGSADNWRSHVKQWQQQCRVVAVDLRNHGRSPHADGMSYAAMAADLEALLDRLGIERAHLLGHSMGGKVAISVARLHPQRVASLIVADIAPQAYGHDHDAVFAGLRRVEQGQPGNRREADALLAEHVEERAVRLFLATNLERDDDGILRLRIGLDEIEGDYDAIMQAPAGEGSYDGPVLVLRGDRSPYVTDAMLPRLHQVLPQAQLVTLENAGHWLHAEQPEAFQAAVNDFLASLEA

SEQ ID NO: 3 - DNA, 23 bp, Artificial Sequence
  Polymerase Chain Reaction primer
  misc_feature (1)..(23): n is A or T or G or C;     r is A or G
gggccatagcatgggnggnaarg

SEQ ID NO: 4 - DNA, 23 bp, Artificial Sequence
  Polymerase Chain Reaction primer
  misc_feature (1)..(23): r is A or G
ggttgatcggcatgcarccartg